(19) United States
(12) Patent Application Publication
(10) Pub. No.: US 2008/0312675 A1
(43) Pub. Date: Dec. 18, 2008

(54) SYSTEM AND METHOD FOR CALCULATING LIMBAL RELAXING INCISIONS

(75) Inventors: Ben Newcott, Costa Mesa, CA (US); Thomas Kim, Irvine, CA (US)

Correspondence Address: ADVANCED MEDICAL OPTICS, INC., 1700 E. ST. ANDREW PLACE, SANTA ANA, CA, 92705, US

(73) Assignee: Advanced Medical Optics, Inc., Santa Ana, CA (US)

(21) Appl. No.: 11/764,724

(22) Filed: Jun. 18, 2007

(51) Int. Cl. A61F 9/00; A61B 19/00; G06F 3/00

(52) U.S. Cl. 606/166; 128/898; 715/700

(57) ABSTRACT

A method and system for calculating ocular incision positions to address astigmatism in an eye. The present design includes providing biometric information, determining an incision location:and angle based on the biometric information and a nomogram such as a Donnenfeld nomogram, relating astigmatism conditions and incision conditions, and presenting the incision location and angle to a user, such as via a graphical user interface. The design is intended to be employed on a general purpose computer and the utility employed, an LRI (Limbal Relaxation Incision) calculator utility may be executed to compute the desired results based on a set of eye measurement inputs.

### BACKGROUND OF THE INVENTION

1. Field of the Invention

The present invention relates generally to the field of medical systems, and more specifically to a mechanized calculation utility for determining Limbal Relaxing Incisions for use by a medical practitioner in performing an eye procedure or surgery.

2. Description of the Related Art

Today's surgeons perform a variety of eye procedures and surgeries, such as a modification of astigmatic keratotomy (AK) using limbal relaxing incisions (LRIs), to treat or correct a patient's astigmatism condition. LRIs used in AK procedures require highly accurate calculations for precise incisions). Typically, before performing an AK procedure, the medical practitioner or surgeon manually calculates the location of a proposed incision and other relevant measures before performing the actual incision.

Practitioners typically use LRIs in the treatment of low to moderate amounts of astigmatism. A surgeon treating astigmatism using LRIs may begin by making a small relaxing incision in the limbus. This incision enables the cornea shape to become more rounded. LRIs are typically located at the outlying edge of the cornea. Today, an LRI procedure may be performed in conjunction with other surgical and laser vision correction procedures. Medical practitioners currently use LRIs as a further means for preventing surgically induced astigmatism following a clear corneal cataract surgery.

A surgeon preparing to perform LRIs typically uses a marker to establish the LRI axis together with manual tools such as a LRI degree gauge to make limbus marks on the eye for cord length (typically ranging from 6-8 mm). These marks are temporary and used for locating where the surgeon will make the incisions. The locations are based upon a formula taking into account the patient's prescription, age and the amount of correction required.

Today's LRI operations typically require the surgeon to make a judgment as to incision length, depth, size, and incision angle based on conditions of the eye encountered during a medical procedure for reducing astigmatism for a patient having a particular profile, such as the aforementioned age, prescription, amount of correction required, and so forth. Once the procedure begins, the surgeon can assess the incisions required based on his or her experience and expertise. However, precise ocular values for the individual patient are not readily available to the surgeon or the patient prior to surgery. As a result, physicians can be placed in a position of discussing the proposed surgery without being able to outline the extent of the procedure necessary, proposed recovery time, and asking for the trust of the patient, who may have a great deal of anxiety due to the uncertainty of the medical procedure. Other medical personnel may not have the calculations readily available either, so everyone entering the ocular surgical theater does not know the number or angle of incisions until the operation begins.

While performing LRI calculations has been generally suggested in the past, no readily available source of incision values, such as number and angle, have been available.

Current techniques for determining the number of incisions, size of incisions, and other parameters associated with LRI surgery can be challenging to calculate in a dynamic environment, such as in a surgical operating theater. Such calculations require time, first learning the patient's ocular parameters used in the manual calculations and use or application of a related nomogram. Further, making such calculations can be inefficient and time consuming to employ during a medical procedure and may be prone to inaccuracy when computed manually under ocular surgical conditions.

Based on the foregoing, it would be advantageous to provide a mechanized calculation utility for use in determining relevant parameters for each incision required to correct an astigmatism that overcomes the foregoing drawbacks present in previously known manual procedures used in preparing for eye procedures involving LRI.

### SUMMARY OF THE INVENTION

According to one aspect of the present design, there is provided a method, configured for operation on a general purpose computer, for calculating ocular incision positions to address astigmatism in an eye. The present design includes providing biometric information, determining an incision location and angle based on the biometric information and a nomogram, such as a Donnenfeld nomogram, relating astigmatism conditions and incision conditions, and presenting the incision location and angle to a user, such as via a graphical user interface. The design is intended to be employed on a general purpose computer and the utility employed, an LRI (Limbal Relaxation Incision) calculator utility may be executed to compute the desired results based on a set of eye measurement inputs.

According to another aspect of the present design, there is provided a system configured to perform ocular incision calculations. The system comprises a computing device comprising a Limbal Relaxation Incision (LRI) calculator utility and a user interface configured to obtain information from a user and interface with the LRI calculator utility to present the user with at least one potential incision. The LRI calculator utility is configured to calculate LRIs based on a nomogram relating astigmatism conditions to incisions.

These and other advantages of the present invention will become apparent to those skilled in the art from the following detailed description of the invention and the accompanying drawings.

### BRIEF DESCRIPTION OF THE DRAWINGS

The present invention is illustrated by way of example, and not by way of limitation, in the figures of the accompanying drawings in which:

FIG. 1A is a functional block diagram of a Internet enabled LRI calculator system that may be employed in accordance with an aspect of the present design;

FIG. 1B illustrates the input parameters required to perform LRI calculations without phacoemulsification;

FIG. 1C illustrates the input parameters required to perform LRI calculations with induced phacoemulsification astigmatism;

FIG. 2 is a flowchart illustrating calculating intermediate values for parameters used in determining the number of LRIs and each associated incision angle, without induced phacoemulsification astigmatism;

FIG. 3A is a flow chart illustrating calculating intermediate values for Steepk Loc High and SteepK Loc Low with respect for how much to treat a patient, without phacoemulsification;

FIG. 3B is a flow chart illustrating auto-calculating intermediate values for FlatK Loc High and FlatK Loc Low with respect for how much to treat a patient, without phacoemulsification;

FIG. 4 is a flow chart illustrating calculating LRIs and intermediate values for Treat and Degrees with respect for incisions on a steep axis, without phacoemulsification;

FIG. 5 is a flowchart illustrating calculating intermediate values for parameters used in determining the number of LRIs and each associated incision angle, with phacoemulsification induced astigmatism;

FIG. 6A is a flowchart illustrating calculating an intermediate value for Steep, with phacoemulsification induced astigmatism;

FIG. 6B is a flowchart illustrating calculating an intermediate value for Flat, with phacoemulsification induced astigmatism;

FIG. 7 is a flow chart illustrating calculating intermediate values for Check Angle and Astigmatism Neutral with respect for phacoemulsification-induced astigmatism;

FIG. 8 is a flow chart illustrating calculating an intermediate value for Delta SteepK with respect for impact of phacoemulsification;

FIG. 9 is a flow chart illustrating calculating an intermediate value for Delta FlatK with respect for impact of phacoemulsification;

FIG. 10 is a flow chart illustrating calculating intermediate values for New SteepK, New FlatK, and Treat with respect for how much to treat a patient with phacoemulsification;

FIG. 11 is a flow chart illustrating calculating an: intermediate value for Degrees with respect for incisions on a steep axis with phacoemulsification;

FIG. 12 is a data diagram illustrating a Donnenfeld. Nomogram for use in determining Slope for both arrangements the with and without phacoemulsification-induced astigmatism;

FIG. 13 is a flow chart illustrating:calculating LRIs and each associated incision angle with respect for incisions on a steep axis without phacoemulsification;

FIG. 14 is an example of operational activity flow that may be supported by a user interface device;

FIG. 15A is a diagram illustrating an example graphical user interface for use to input patient data to the LRI calculator; and

FIG. 15B is a diagram illustrating an example graphical user interface for use by the LRI calculator to output patient results.

### DETAILED DESCRIPTION OF THE INVENTION

The following description and the drawings illustrate specific embodiments sufficiently to enable those skilled in the art to practice the system and method described. Other embodiments may incorporate structural, logical, process and other changes. Examples merely typify possible variations. Individual components and functions are generally optional unless explicitly required, and the sequence of operations may vary. Portions and features of some embodiments may be included in or substituted for those of others.

The present design is directed to an accurate, reliable, and efficient means for LRI calculations for use in performing a corrective procedure to mitigate a patient's astigmatism condition.

The present design provides an LRI calculator system that may show where and how long to make limbal relaxing incisions for reducing a patient's astigmatism via a user interface based on a nomogram, patient keratometry (K) measurement readings, and other biometric factors such as if phacoemulsification-induced astigmatism is involved. The calculator system may present data and information via a graphical user interface (GUI) to show marks or indicate each incision location superimposed on top, for example using an image overlay, of a real-time image of the patient's eye. The nomogram may incorporate rules and assumptions that describe how astigmatism behaves and reacts to LRIs.

The calculator system may be used to accurately determine the number and location of each incision and relate information regarding incision angle required to correct a patient's eye aliment or condition. The system may generate highly accurate and repeatable calculations enabling a surgeon to precisely locate each required incision site. The operable range of the present design may enable surgeons to calculate LRIs for a greater range of measurement values on the eye for cord length than achievable with current manual measurement methods.

The present design may provide a quick, easy to use, and reliable LRI calculation utility flexible enough to tailor the calculations for eye correction based-on whether or not astigmatism was phacoemulsification induced.

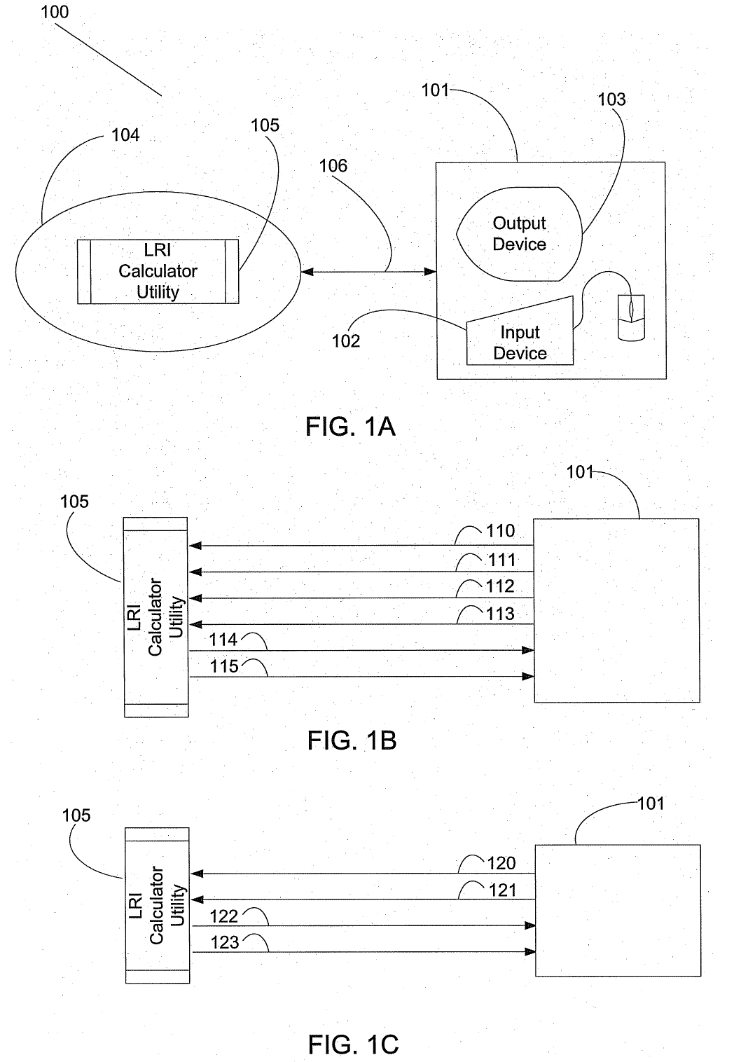

While the present design may be used in various environments and applications, it will be discussed herein with a particular emphasis on a medical or hospital environment, where a surgeon or health care practitioner performs. Alternatively, one embodiment of the present design is an LRI calculator system accessible from the Internet using either a personal computer, personal digital assistant, web enabled cell phone, and other browser enabled devices capable of interacting with the calculator system. A functional block diagram of an exemplary LRI calculation system 100 for determining relevant parameters regarding surgical decisions that may be employed in accordance with an aspect of the present invention is illustrated in FIG. 1A. A browser enabled device 101, for example a personal computer, may provide a user interface and may comprise an input device 102 and output device 103 such as a QWERTY keyboard with mouse, and LCD display screen, respectively. An Internet enabled LRI calculator system 104 may include a LRI calculator utility 105, or “web-hosted system”, and may comprise hardware, firmware, and software necessary to realize LRI calculations and the functionality discussed below based on operator/user submitted information relating a patient's ocular biometrics. Communications network 106 may provide an access mechanism and connection path for operators/users who desire to access LRI calculator utility 105.

While the present design may be described as an Internet enabled, or web deployed, software application capable of supporting multiple users simultaneously. The present design, may be realized in, for example, a personal computer, cell phone or personal digital assistant (PDA) application, or distributed on physical media such as compact disc, and combinations thereof, is illustrated herein in an exemplary web deployed implementation. It is to be understood that any surgical eye and laser vision correction procedure requiring LRIs to be determined for correcting an eye condition may benefit from the design presented herein. As such, the present design may store, retrieve, transmit, and employ values using storage devices, processors, and other devices known in the art to provide the functionality described herein. For example, intermediate calculated values may be stored or maintained in RAM or some form of flash memory, and various databases may be employed. The functionality described herein may entail performing tasks over various devices, and the functions described are not necessarily performed on a single device, such as on a single processor or ASIC or other known device.

Nomogram

The nomogram employed in the present design is called a Donnenfeld nomogram, named after its creator, Dr. Eric Donnenfeld. Other nomograms may be employed in accordance with the present design, but the Donnenfeld nomogram will be described in detail here. In general, the Donnenfeld nomogram is based on operative experience that works extremely well for calculating LRIs to correct residual refractive error.

A Donnenfeld nomogram determines the number of LRI incisions and the total degrees of each incision. The Donnenfeld nomogram may include a table of data to affect a graphical function that relates the degree or amount of a patient's astigmatism relative to the total incision clock hours (nomogram). The Donnenfeld nomogram may approximate total incision clock hours (linear) from the total incision clock hours (nomogram) data by calculating the slope 212 of the graphical line formed by plotting astigmatism 1201 versus total incision clock hours (nomogram).

Simply put, the Donnenfeld nomogram employs two input scales of known values for astigmatism and total incision clock hours (nomogram) and one output scale where a resultant rounded incision size is made available.

The Donnenfeld nomogram for LRIs provides, in the case involving 0.50 D of cylinder, one incision of 1.5 clock hours; and for cases of 0.75 D, 1.50 D, or 3.00 D of cylinder, two paired incisions of one clock hour, two clock hours, or three clock hours, respectively.

The Donnenfeld nomogram may suggest lengthening the incisions slightly for patients exhibiting against-the-rule astigmatism and younger patients who are less than 45 years old. Patients over 65 years old may require shorten incisions. The nomogram may be adjusted to reflect a surgeon's choice: of different lasers or instruments.

A simplified version of the Donnenfeld nomogram for LRIs is presented in Table 1.

TABLE 1

| Preoperative | Number of | Length of Incisions |
|---|---|---|
| Astigmatism | Incisions | (Clock Hours) |
| 0.50 D | 1 | 1.5 |
| 0.75 D | 2 | 1 |
| 1.50 D | 2 | 2 |
| 3.00 D | 2 | 3 |

All incisions are placed 0.5 mm from the limbus in the correct axis. With respect to length of incisions, patients who have against-the-rule astigmatism or who are less than 45 years old may benefit from slightly longer incisions Shorter incisions may be indicated for patients older than 65 years. Regarding preoperative astigmatisms of 3.00 D, LRIs can be used to correct up to 3.00 D of astigmatism if a laser correction is contraindicated for financial or medical reasons.

Nomogram Implementation.

FIG. 1B illustrates the biometric input parameters required to adequately describe a patient's eye condition, entered by a surgeon or other practitioners, sufficient to generate LRI calculations, without phacoemulsification-induced astigmatism, in accordance with an aspect of the present invention. The operator/user may enter values for a patient's K readings, and may include but is not limited to: SteepK 110, FlatK 111, and SteepK Loc 112. SteepK represents a steep corneal value, FlatK a flat corneal value, SteepK Loc a steep corneal location, and FlatK Loc a flat corneal location. The present design may employ any or all of these input parameters to calculate LRIs and may respond to the given input by generating output values for the actual incisions or LRIs 114 and each associated Incision Angle 115. The terms Steep Merdian K, and Steep Merdian, and SteepK Loc are generally used interchangeably in this document. Similarly, the terms Flat Merdian K, Flat Merdian, and FlatK Loc are used interchangeably herein. This terminology is used to assess where particular values com from and go when being used by the present tool.

FIG. 1C illustrates the biometric input parameters required to adequately describe a patient's eye condition, entered by a surgeon or other practitioner, sufficient to generate LRI calculations when combined with phacoemulsification-induced astigmatism, in accordance with an aspect of the present design. The operator/user may enter values including but is not limited to SIC 120, indicating the degree of RK induced hyperopia, and Incision location 121 indicating the location of the main incision. The present design may employ either or both of these input values to calculate LRIs and may respond to the given input by generating output parameters for LRIs 122 and each associated Incision 123.

The biometric input K readings SteepK (steep corneal value). FlatK (flat corneal value), SteepK Loc (steep corneal location), FlatK Loc (flat corneal location), Induced, and Location are understood by those skilled in the RK art. The biometric output values for LRIs and each associated Incision Angle is also understood by those skilled in the art.

### Calculator Utility Operations Without Phacoemulsification-Induced Astigmatism

The present design may include a LRI calculator utility 105 configured to determine biometric surgical values for a patient's total number of LRIs and associated incision angles for medical eye surgeries and procedures in the arrangement where phacoemulsification-induced astigmatism is not involved.

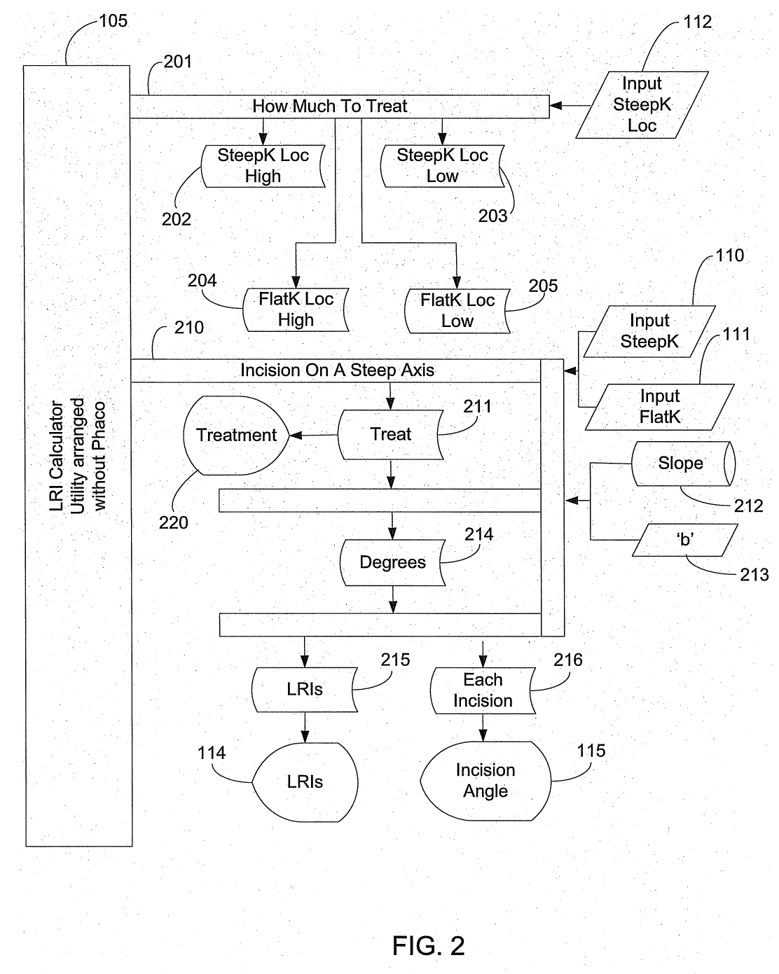

FIG. 2 illustrates an exemplary LRI calculator utility 105 that first determines how to treat a patient and subsequently calculates incisions on a steep axis when phacoemulsification-induced astigmatism is not present. The LRI calculation system 100 may determine and graphically show (via user interface) where and how:long to make limbal relaxing incisions for reducing a patient's astigmatism. The user interface generates output values for LRIs 114 and associated Incision Angle 115 when provided input values of SteepK 110, SteepK Loc 112, FlatK 111, Slope 212, and “b”, 213 as illustrated in FIG. 2. “b” is a constant which can vary in value and depends on various circumstances, but typically represents the y-intercept of the Donnenfeld nomogram, used to determine the LRI amounts for discrete values of astigmatism. A representative value for “b” is −0.33333333. In this arrangement, the present design may determine parameter values that relate information relevant to How-Much-To-Treat 201 a patient's condition presented in terms of SteepK Loc High 202, SteepK Loc Low 203, FlatK Loc High 204, and FlatK Loc Low 205. In addition, the present design may determine parameter values relevant to performing a surgical Incision-On-A-Steep-Axis 210, presented in terms of Treat 211 and Degrees 214 as intermediate parameter values for use as input to further calculations.

The amount of correction necessary to mitigate a patient's condition may be computed by generating the number of LRIs 215, and Each Incision 216 for use by a surgeon while performing an eye procedure. The design can present patient information to the surgeon, via a graphical user interface (GUI) or other suitable method that fulfills the purposes of a GUI, including but not limited to treatment 220, the number of LRIs 114 and associated Incision Angle 115, and may:show marks or indicate each incision location superimposed over a real-time image of the patient's eye. A surgeon may use the information presented by LRI calculation system 100 to ascertain the amount of correction needed to mitigate the patient's eye condition while performing an ocular procedure or surgery.

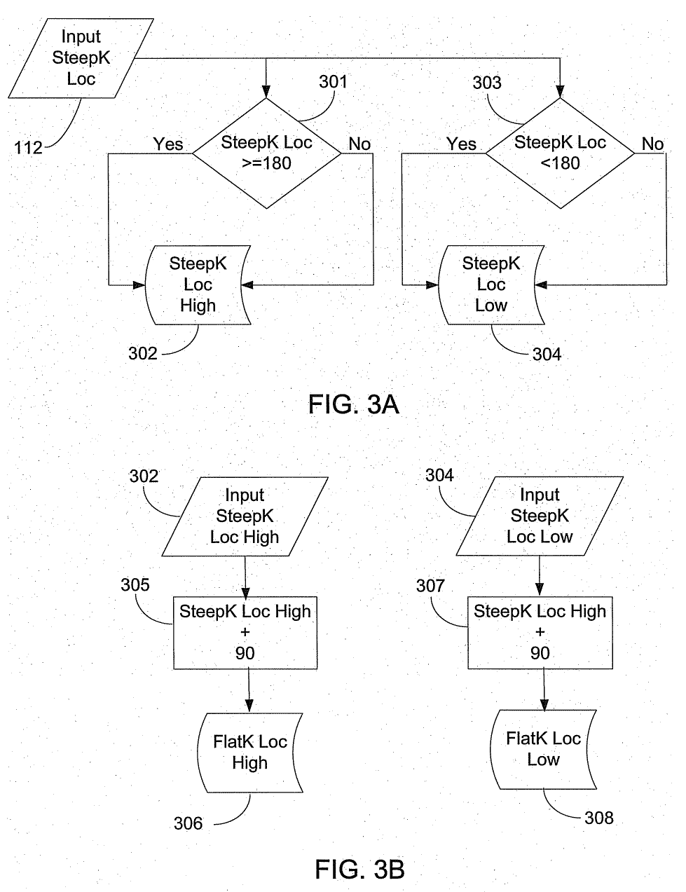

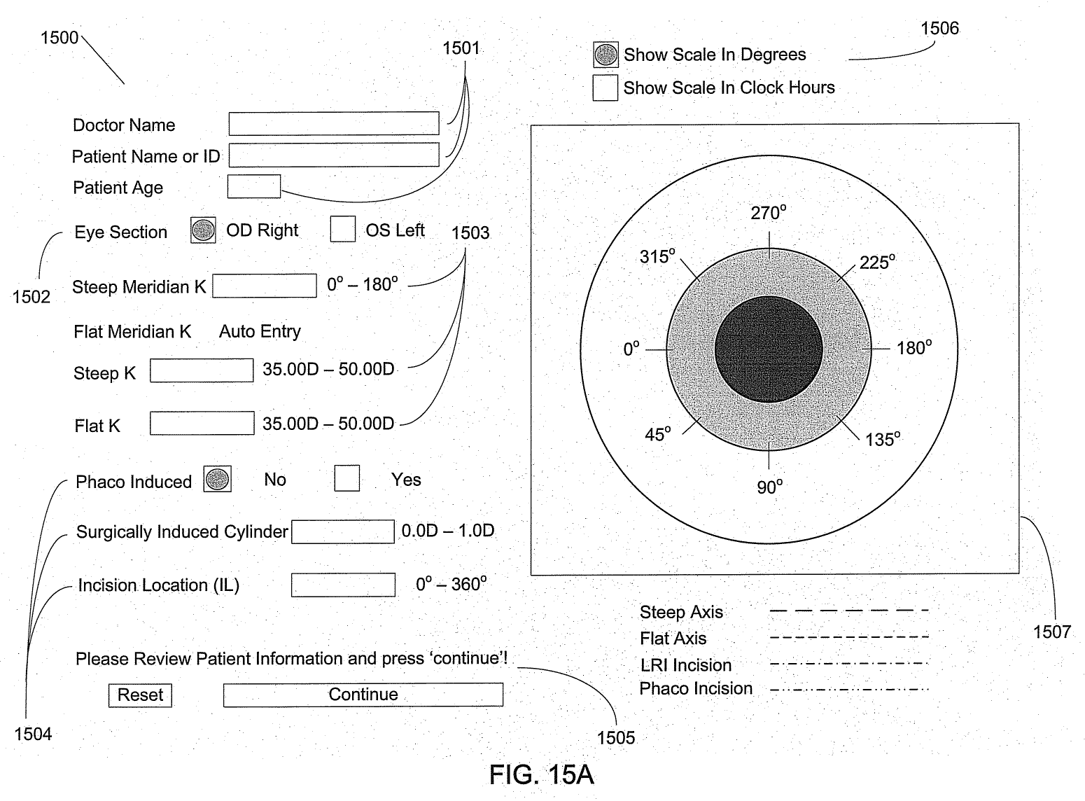

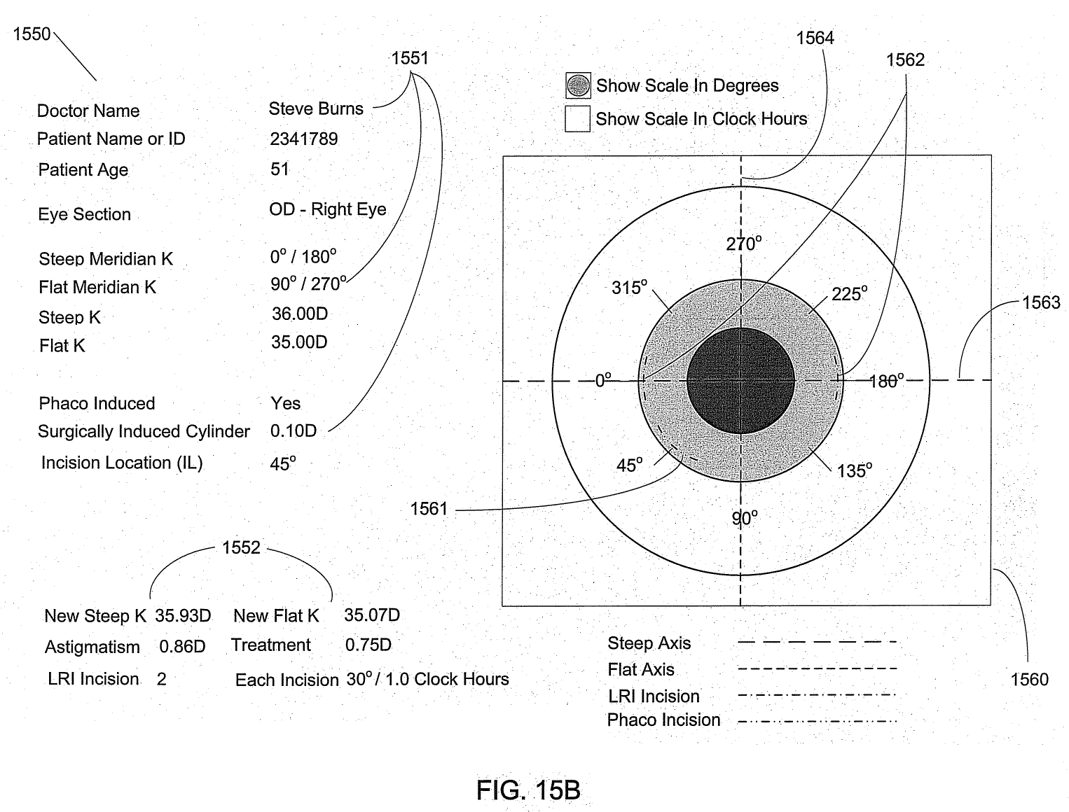

FIG. 3 illustrates calculating How-Much-To-Treat 201, for the “without phacoemulsification-induced astigmatism” condition. The primary objective of the actions of FIG. 3 is to determine the location of the Steep Axis and Flat Axis and may present these axes to the surgeon via the GUI (as shown in FIG. 15B).

The LRI calculator utility 105 may determine SteepK Loc High 302 by evaluating SteepK Loc 112 at decision point 301. If decision point 301 is greater than or equal to 180 degrees, then the present design may set SteepK Loc High 302 equal to SteepK Loc 112. If decision point 301 is less than 180 degrees, SteepK Loc High 302 is set equal to SteepK Loc 112 plus 180 degrees.

The present design may determine SteepK Loc Low 304 by evaluating SteepK Loc 112. If decision point 303 contains a value less than 180 degrees, then the present design may set SteepK Loc Low 304 equal to SteepK Loc 112. If decision point 303 is greater than or equal to 180 degrees, then SteepK Loc Low 304 can be set equal to the quantity SteepK Loc 112 minus 180.

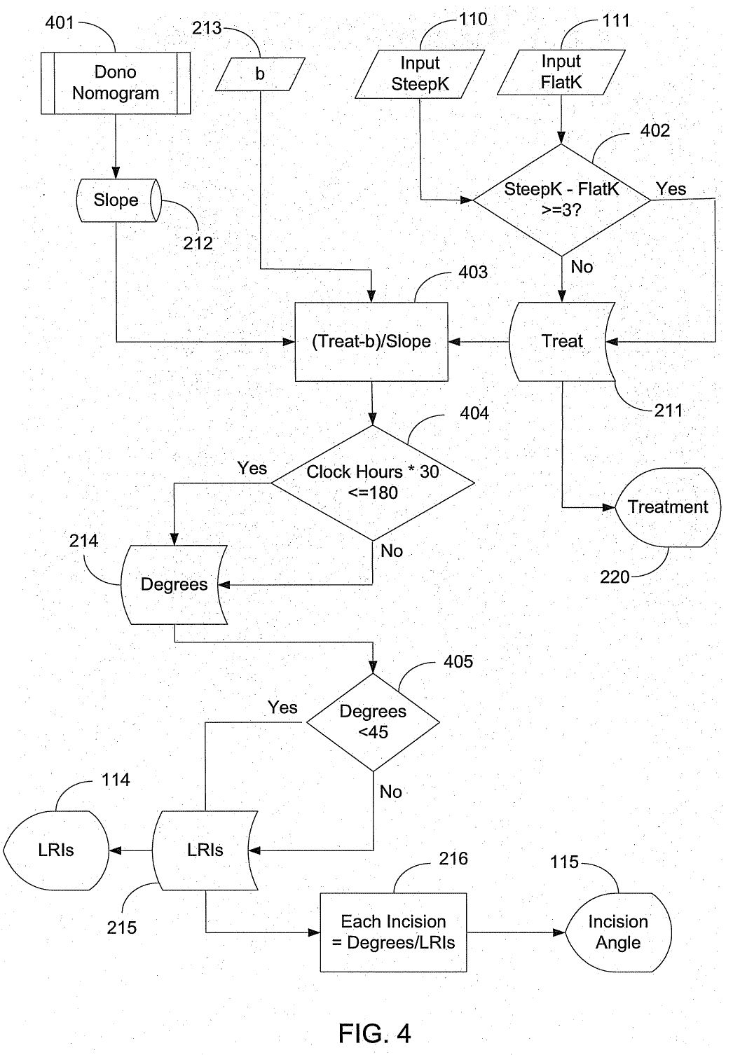

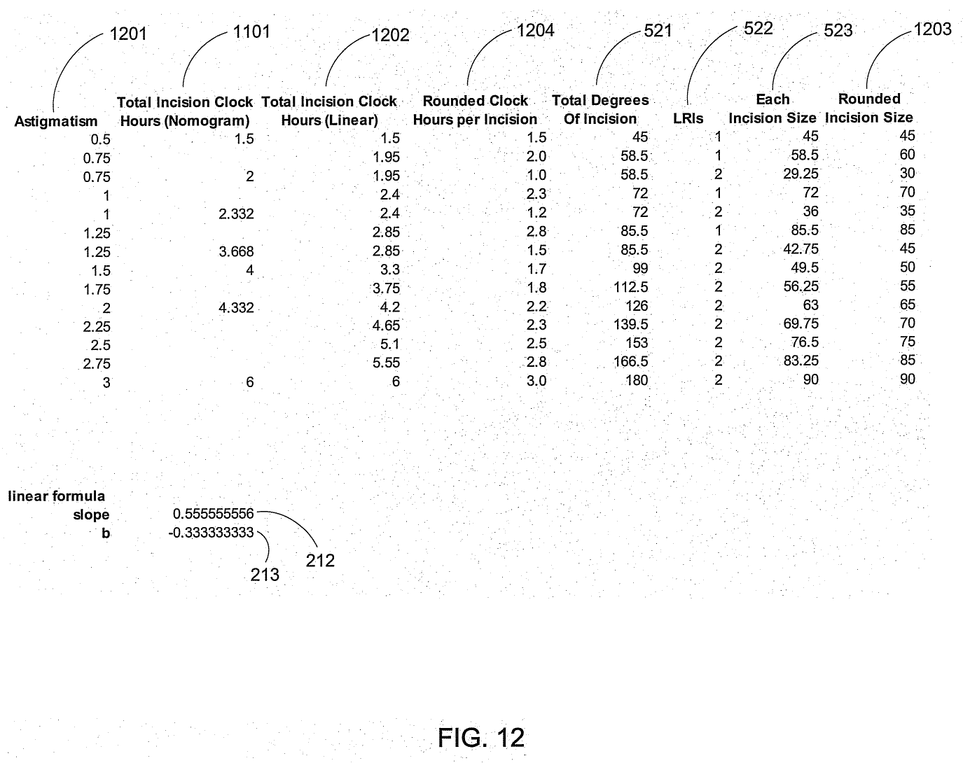

FIG. 3B computes compliance with boundary conditions for FlatK Loc High 306 and FlatK Loc Low 307. In this arrangement, the present design may rotate the flat axis ninety (90) degrees from the steep axis. The present design may determine FlatK Loc High 306 by evaluating SteepK Loc High 302 at decision point 305. FlatK Loc High 306 is set equal to SteepK Loc High 302 plus 90 degrees. In a similar manner, the present design may determine FlatK Loc Low 308 by evaluating SteepK Loc Low 304 at decision point 307. FlatK Loc Low 308 is set equal to SteepK Loc Low 304 plus 90 degrees. FIG. 4 illustrates calculating Incisions-On-A-Steep-Axis 210 for the “without phacoemulsification-induced astigmatism” condition. The system may generate output values for the number of LRIs 215 and each associated Incision Angle 216. In order to generate output values, the present design may calculate values for Treat 211, Clock Hours 404, and Degrees 214 as intermediate parameter values. Treat 211 represents the degrees of astigmatism to be treated, and if beyond a certain predetermined value, the astigmatism may be untreatable and the present design may present this treatment value at point 220 to the surgeon via the GUI. Clock hours 404 represents the position, on a clock, of the incision, ranging from 0 to 12, convertible to Degrees 214, ranging from zero to 360. FIG. 4 illustrates the methods input values for use in calculating of LRIs 215 and each Incision Angle 216 the Incisions-On-A-Steep-Axis 210 arrangement: Slope 212, ‘b’ 213 (a constant), SteepK 110, and FlatK 111. In this arrangement, the present design may employ a nomogram, for example a Donnenfeld Nomogram 401, and realize linear values for Slope 212 based on the underlying nomogram assumptions, equations, formulas, and rules that describe how an astigmatism may behave and react to LRIs as illustrated in FIG. 12. Use of a Donnenfeld Nomogram 401 may enable support over a range of astigmatism values such as illustrated at point 1201. The Donnenfeld Nomogram 401 may realize total nomogram based incisions in terms of Clock Hours as illustrated at point 1101. Determining total linear incision clock hours at point 1202 may be realized by solving the formula of Equation (1):

TLI:Clock Hrs=(Astigmatism−‘b’)/Slope (1)

Again, “b”, is the y-axis intercept for the linear formula of the Donnenfeld nomogram and is used to determine the LRI values for discrete values of astigmatism.

Degrees 521 are correlated to Clock Hours using Equation (2):

Degrees=Clock Hours*30 (2)

FIG. 12 illustrates sample output results for the total number of LRIs 522 and Each Incision 523 as generated by LRI calculator utility 105. The calculations may involve employing the following formula to determine Each Incision 523 where:

Each Incision=Degrees/LRIs (3)

The LRI calculator utility 105 may determine Rounded Incision Size 1203, from the value for Each Incision 523 resulting from equation (3), by rounding up the value for Each Incision 523 to the nearest 5 (five) degrees. Rounded Clock Hours Per Incision 1204 may be obtained by taking the Rounded Incision Size 1203 value and dividing this value by 30, in order to convert from degrees to clock hours, and rounding-up the result to one place after the colon point delineating hours from minutes.

Rounded Incision Size 1203 may involve calculation of the Phaco Incisions ranging: from 0.1-1.0, and rounded to the nearest 0.1 D. In addition, Rounded Incision Size 1203 may include calculations when only one opposite. LRI is possible when having a Phacoemulsification Incision on Steep-Axis. The present design may restrict to have only a maximum 90° incision, resulting in a treatment up to 1.25 Diopters.

The present design may present values Rounded Incision Size 1203 and Rounded Clock Hours Per Incision 1204 to the surgeon via the GUI.

The present design may employ a data diagram, such as the Donnenfeld Nomogram 401 illustrated in FIG. 12, configured to determine values, such as linear Slope 212 when a phacoemulsification-induced astigmatism is not present. Input may be the Donnenfeld Nomogram 401 values, calculating the slope of an incision line according to the following formula:

Slope=(Largest Astigmatism Value−Smallest Astigmatism Value)/(Largest Total Incision Clock Hours (Nomogram)−Smallest Total Incision Hours) (4)

Decision point 402 may compute Treat 211 by solving the formula:

Treat=SteepK minus FlatK (5)

If decision point 402 yields a value that is greater than or equal to a predetermined value (degrees of astigmatism), for example three, Treat 211 may be set to a default value equal to this predetermined value. If decision point 402 yields a value that is not greater than or equal to the predetermined value, then the present design may set Treat 211 equal to the quantity SteepK minus FlatK.

The LRI calculator utility 105 may determine Clock Hours at point 403 using Treat 211, ‘b’, 213, and Slope 212 using the formula:

Clock Hours=(Treat−b)/Slope (6)

Again, the LRI calculator utility 105 may determine Degrees 214 at point 404 using the formula:

Degrees=Clock Hours*30 (7)

Boundary conditions are then verified: if the value for Clock Hours at point 403 is less than or equal to 180 degrees. Degrees 214 is set equal to Clock Hours multiplied by 30. It the value for Clock Hours at point 403 is greater than 180 degrees, then the present design may set Degrees 214 equal to 180 degrees.

LRI determining point 215 may evaluate Degrees 214 at decision point 405. If the value received at decision point 405 is less than 45 degrees, then LRIs may be set equal to 1. If greater than 45 degrees, then LRIs are set equal to 2.

The LRI calculator utility 105 may determine each Incision Angle 216 by solving Equation (8):

Incision Angle=Degrees/LRIs (8)

The LRI calculator utility 105 may present output values for LRIs 114 and each associated Incision Angle 115 via a user interface for a surgeon's use in conducting a procedure.

The LRI calculator utility 105 may determine a value for Astigmatism and present the result at 1552. Astigmatism is set equal to SteepK 110 minus FlatK 111 for the non-phacoemulsification configuration. The present design may round the resulting astigmatism value to the next quarter (in diopters).

The calculator system 100 may present patient information to the surgeon, via a graphical user interface (GUI) or other suitable method that fulfills the purposes of a GUI, including but not limited to the number of LRIs 114, and associated Incision Angle 115. The calculator system may present data and information via GUI to show marks or indicate each incision location, and these markings may be superimposed over a real-time image of the patient's eye or a generic eye. A surgeon may use the information presented by LRI calculation system 100 to ascertain the amount of correction necessary to mitigate the patient's eye condition while performing an ocular procedure or surgery.

### Calculator Utility Operations With Phacoemulsification-Induced Astigmatism

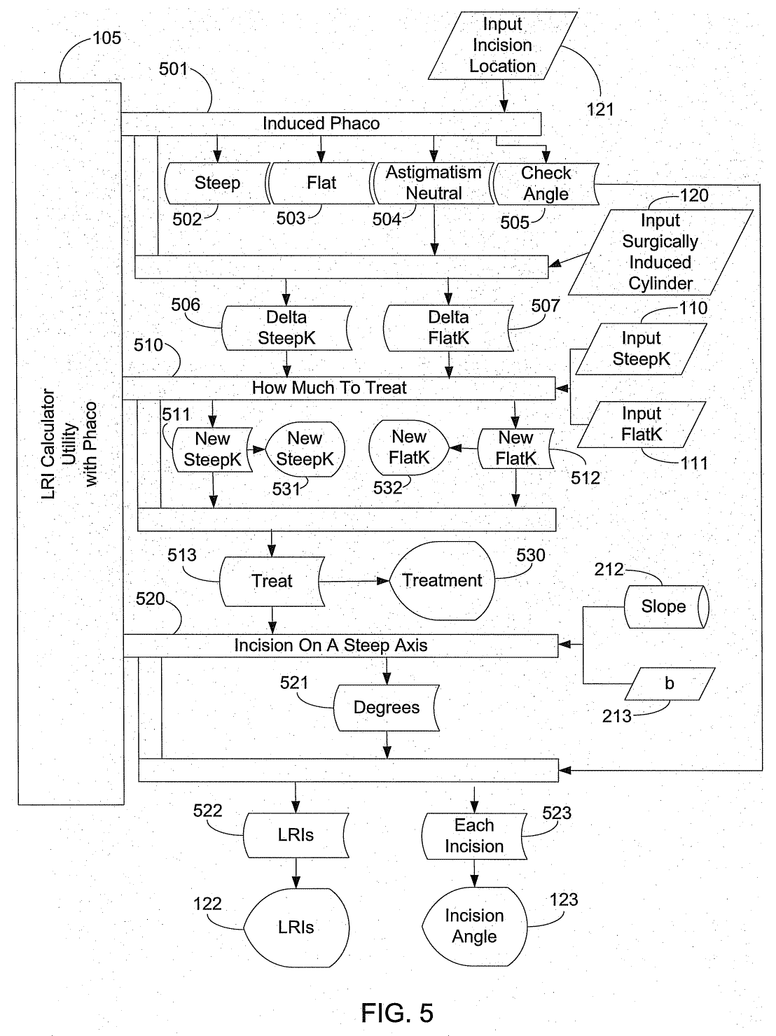

FIG. 5 illustrates an LRI calculator utility 105 that determines how much to treat a patient and computes incisions on a steep axis with a phacoemulsification-induced astigmatism. The LRI calculator utility 105 may show where and how long to make limbal relaxing incisions for reducing a patient's astigmatism via a user interface by generating output values for LRIs 122 and associated Incision Angle 123.

In the phacoemulsification-induced astigmatism arrangement, the present designs method may evaluate the effect of the phacoemulsification incision on the astigmatism and may employ vector analysis to calculate the following intermediate values for Induced-Phaco 501: Steep 502, Flat 503, Astigmatism Neutral 504, and Check Angle 505. Vector analysis may account for the induced astigmatism resulting from phacoemulsification and the vector force may be applied to the steep and flat K's in order to determine values for Delta SteepK and Delta FlatK.

FIG. 5 illustrates using Incision Location 121 as an input value to calculate the above intermediate values. If the astigmatism is neutral, the system may use Surgically Induced Cylinder 120 as a value to determine additional intermediate parameter values for Delta SteepK 506 and Delta FlatK 507.

The system may calculate intermediate values for How-Much-To-Treat 510, such as different K values including New SteepK 511, New FlatK 512, and Treat 513. FIG. 5 illustrates the present design using SteepK 110, FlatK 111, Delta SteepK 506 and Delta FlatK 507 as an input values provided by a user, operator, program, or other inputting entity to calculate the above phacoemulsification-induced astigmatism intermediate values.

The present design may determine parameter values that relate information relevant to performing a surgical Incision-On-A-Steep-Axis 520, presented in terms of Degrees 521 (intermediate value), LRIs 522, and Each Incision 523 (system output values). The present design may employ, as input; parameter values for Slope 212, ‘b’ 213, and Treat 513 and determine intermediate value Degrees 521, generally representing a line for incision in degrees. The present design may determine the amount of correction necessary to mitigate a patient's condition based on the value for Degrees 521 by generating the number of LRIs 522, and Each Incision 523.

The LRI calculator utility 105 may present patient information to the surgeon via a GUI or other suitable method that fulfills the purposes of a GUI, including but not limited to Treatment 530, New SteepK 531, New FlatK 532, number of LRIs 122 and associated Incision Angle 123 as output values. The calculator system may present data and information via a graphical user interface (GUI) to show marks or indicate each incision location and may superimpose these markings over a real-time image of the patient's eye. A surgeon may use the information presented by the system to ascertain the amount of correction necessary to mitigate the patient's eye condition while performing an ocular procedure or surgery.

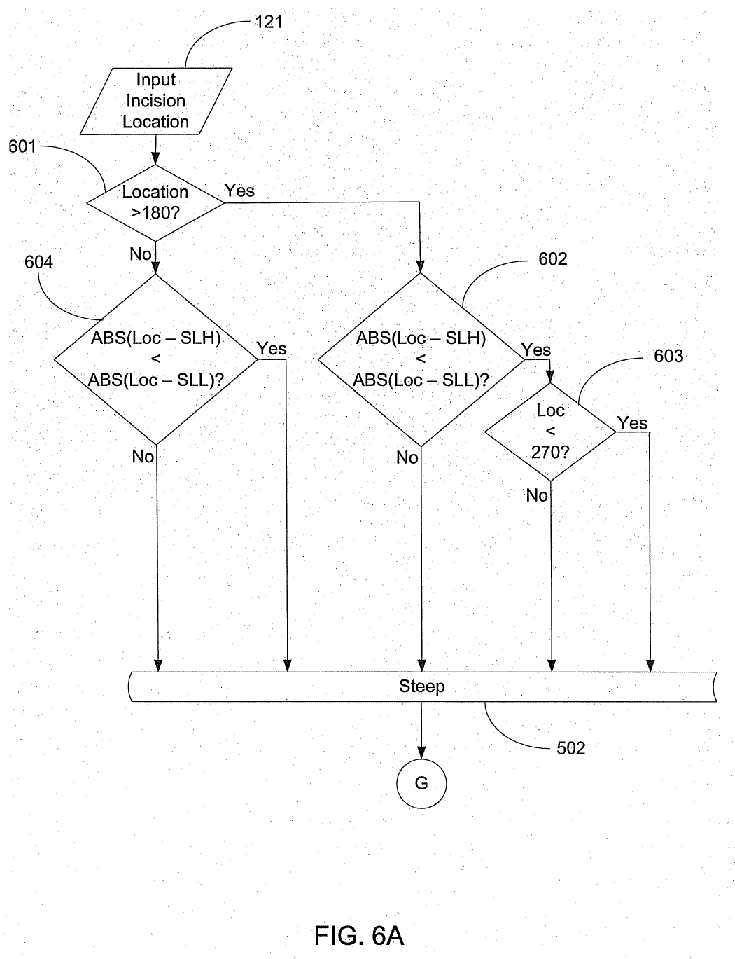

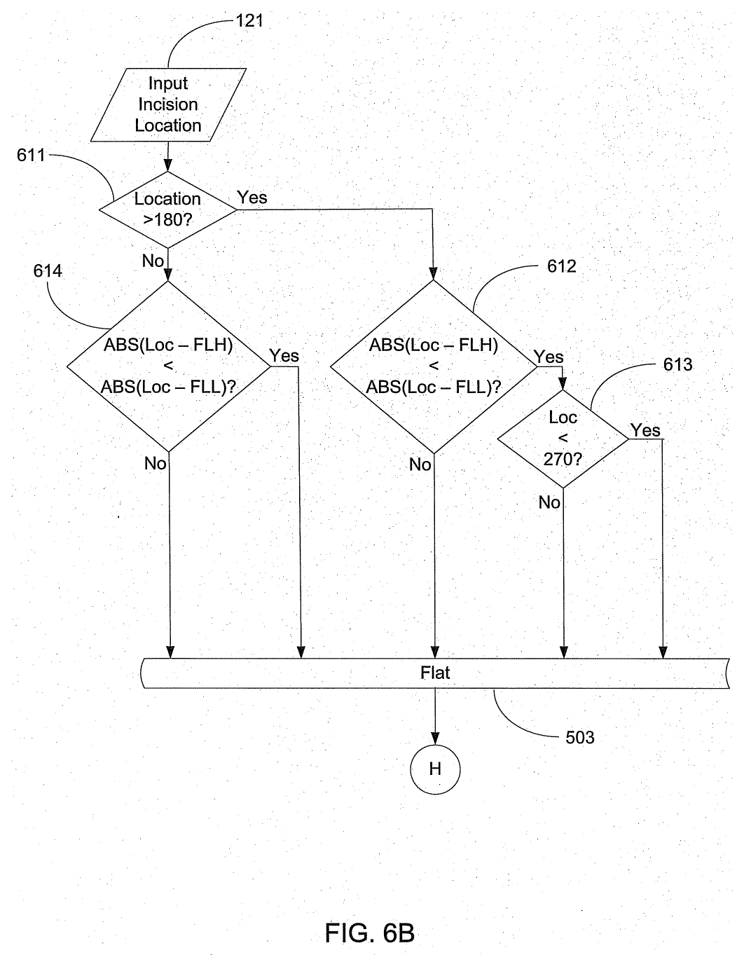

FIG. 6A illustrates calculating Steep 502 relevant to surgically Induced-Phaco 501 condition. FIG. GA illustrates receiving Incision Location 121 as an input value for calculating Steep 502. The LRI calculator utility 105 may determine the value for Steep 502 by evaluating Incision Location 121 at decision point 601. If decision point 601 contains a value greater than or equal to 180 degrees, then the present design may perform a further evaluation at decision at point 602 by evaluating whether the absolute value of (Location minus SteepK Loc High) is less than or equal to the absolute value of (Location minus SteepK Loc Low).

If decision point 602 is true, the LRI calculator utility 105 may perform a further comparison at decision point 603. If decision point 602 is false, the present design may set Steep 502 equal to the quantity (Location minus SteepK Loc High minus 180 degrees), again a boundary condition forced setting if decision point 603 indicates a value less than 270 degrees, Steep 502 is set equal to the absolute value of (Location minus SteepK Loc High). If decision point 603 indicates a value greater than or equal to 270, Steep 502 is set equal to the quantity of the absolute value of (Location minus SteepK Loc High minus 180 degrees). Decision point 601 being less than or equal to 180 degrees causes LRI calculator utility 105 to perform a further comparison at 604 by evaluating whether the absolute value of (Location minus SteepK Loc High) is less than absolute value (Location minus SteepK Loc Low).

If decision point 604 is true, Steep 502 is set equal to the absolute value of (Location minus SteepK Loc High). If false, Steep 502 is set equal to the absolute value of (Location minus SteepK Loc Low).

FIG. 6B illustrates calculating Flat 503 for a surgically Induced-Phaco 501 condition. FIG. 6B illustrates using Incision Location 121 as an input value for calculating Flat 503. Incision Location 121 represents the location of the incision, while Flat 5.03 is the flat part of the cornea. The present design may determine the value for Flat 503 by evaluating Incision Location 121 at decision point 611. This is a boundary condition evaluation. If decision point 611 is a value greater than or equal to 180 degrees, the present design may perform a further evaluation at decision point 612 where the method may evaluate whether the absolute value of (Location minus SteepK Loc High) is less than or equal to the absolute value of (Location minus SteepK Loc Low). If true, the LRI calculator utility 105 may perform a further comparison at decision point 613. If false, then the present design sets Flat 503 equal to the absolute value of (Location minus FlatK Loc Low minus 180) if decision point 613 is less than 270 degrees, Flat 503 is set equal to the absolute value of (Location minus FlatK Loc Low). If greater than or equal to 270 degrees, Flat 503 may be set equal to the absolute value of (Location minus FlatK Loc High minus 180 degrees).

If decision point 611 encounters a value less than or equal to 180 degrees, the system at decision point 614 evaluates whether the absolute value of (Location minus FlatK Loc High) is less than the absolute value of (Location minus FlatK Loc Low).

If decision point 614 contains a value less than 270 degrees, Flat 503 is set equal to the absolute value of (Location minus FlatK Loc High). If decision point 614 receives a value is greater than or equal to 270 degrees, then Flat 503 is set equal to the absolute value of (Location minus FlatK Loc Low).

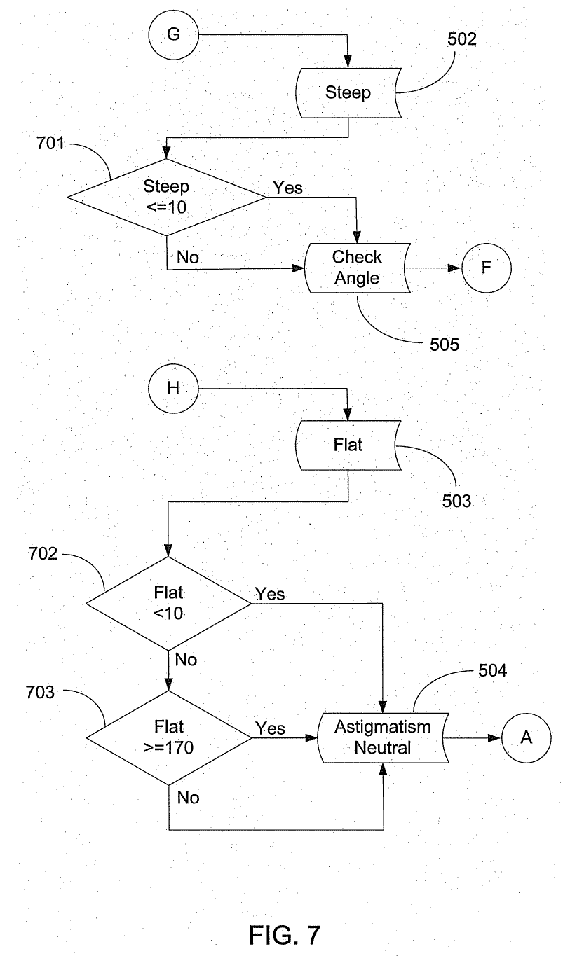

FIG. 7 illustrates calculating intermediate values Check Angle 505 and Astigmatism Neutral 504 for the phacoemulsification-induced astigmatism condition. Previously calculated and stored intermediate value Steep 502 may be used to determine a value for Check Angle 505 at decision point 701. If decision point 701 indicates the angle is within a predetermined number of degrees from a steep angle, then an indication is provided to operate on the steep angle. Thus if the decision point 701 indicates an angle less than or equal to a predetermined angular value, such as 10 degrees, then the LRI calculator utility 105 sets Check Angle 505 equal to 0 (zero), indicating an “operate on steep” condition. If decision point 701 is greater than the predetermined angular value, then Check Angle 505 is set equal to the quantity 180 degrees minus Steep.

The present designs apparatus and method may determine whether the patient's astigmatism is neutral or not. Astigmatism Neutral 504 is determined at decision point 702 by evaluating Flat 503. If decision point 702 indicates a value less than a predetermined amount, such as 10 degrees, the LRI calculator utility 105 may set Astigmatism Neutral 504 indication to “neutral”. If decision point 702 is greater than the predetermined amount, the present design performs a further comparison to determine if the patient's astigmatism is neutral. If Flat 503 is greater than or equal to 170 degrees at decision point 703, Astigmatism Neutral 504 equal to “neutral”. If decision point 703 receives a value less than 170 degrees, then LRI calculator utility 105 may set Astigmatism Neutral 504 equal to “not-neutral”.

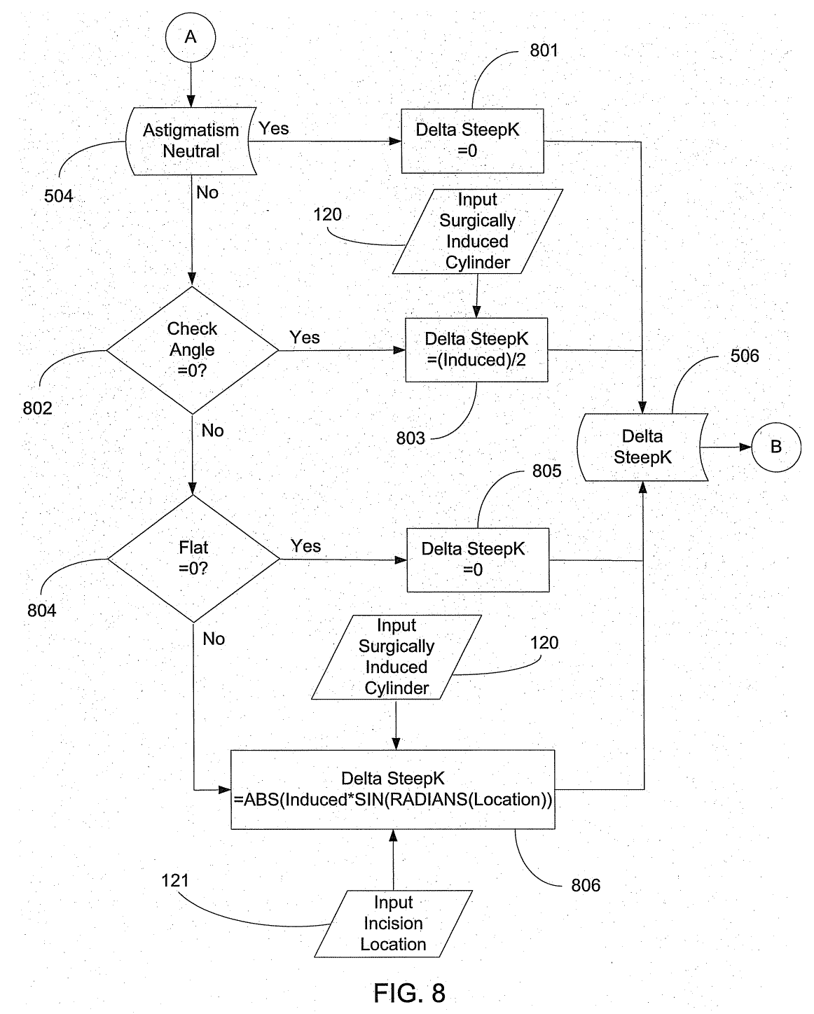

FIG. 8 illustrates using vector analysis to take in account the induced astigmatism resulting from performing phacoemulsification and applies the force to SteepK to determine Delta SteepK. The design calculates Delta SteepK 506 with respect to Induced-Phaco 501 for phacoemulsification-induced astigmatism. FIG. 8 illustrates using the previously determined value for Astigmatism Neutral 504 as an input value and determining the impact of a phacoemulsification wound on Delta SteepK 506. If the value of Astigmatism Neutral 504 is “neutral,” Delta SteepK 506 is set equal to zero at point 801. If the value of Astigmatism Neutral 504 is “not-neutral,” then the system evaluates Check Angle 505 at decision point 802 to determine Delta SteepK 506. If decision point 802 equals zero, then the system uses Surgically Induced Cylinder (SIC) 120 at point 803 to set Delta SteepK equal to the result obtained from:

Delta SteepK=(SIC 120)/2 (9)

If decision point 802 is not equal to zero, then the LRI calculator utility 105 may evaluate Flat 503 at decision point 804 to determine Delta SteepK 506. If decision point 804 equals zero, then the system sets Delta SteepK 506 equal to zero at point 805. If decision point 804 is not equal to zero, then the present design may employ input values Surgically Induced Cylinder 120 and Incision Location 121 at point 806 to set Delta SteepK 506 equal to:

Delta SteepK=absolute value (Induced*Sin[Location (in units of radians)]) (10)

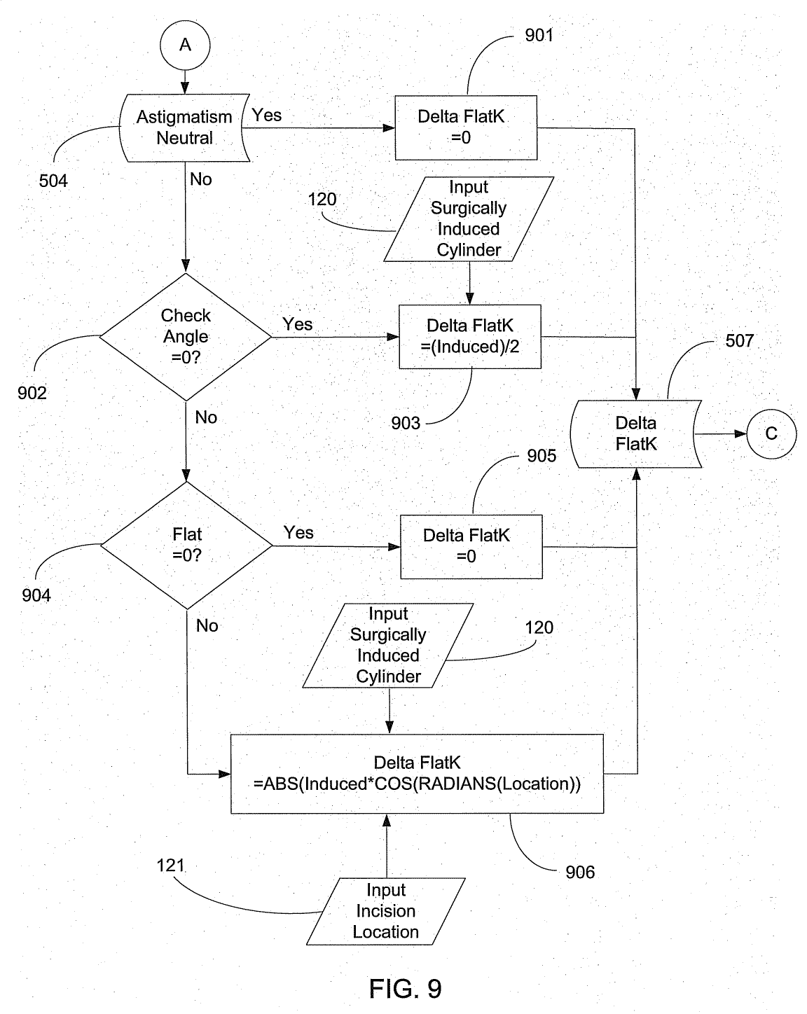

FIG. 9 illustrates employing vector analysis to account for induced astigmatism from performing phacoemulsification 5S and applying the force to FlatK to determine Delta FlatK. The system calculates an intermediate value for Delta FlatK 507 for Induced-Phaco 501. FIG. 9 shows use of Astigmatism Neutral 504 as an input for determining the impact of a phacoemulsification wound on Delta FlatK 507. If the value of Astigmatism Neutral 504 is “neutral,” Delta FlatK 507 is set equal to zero at point 901. If the value of Astigmatism Neutral 504 is “not-neutral,” then the system evaluates Check Angle 505 at decision point 902 to determine Delta FlatK 507.

If decision point 902 contains a value equal to zero, Surgically Induced Cylinder 120 is used at point 903 to set Delta FlatK equal to:

Delta FlatK=(SIC 120)/2 (11)

If decision point 902 is not equal to zero, the system (LRI calculator utility 105) may evaluate Flat 503 at decision point 904 to determine Delta FlatK 507. If decision point 904 contains a value of zero, Delta FlatK 507 may be set equal to zero at point 905. If decision point 904 is not equal to zero, input values SIC 120 and Incision Location 121 may be used at point 906 to set Delta FlatK 507 equal to:

Delta FlatK=absolute value (Induced*Cos[Location (in units of radians)]) (12)

If the values for Surgically Induced Cylinder (astigmatism 1201) ranges from 0.1 to 0.5 diopters, the present design may set the total degrees of incision 521 to thirty degrees. For astigmatism 1201 values ranging between 0.5 to 1.0 diopters, the present design may set the total degrees of incision 521 to forty degrees.

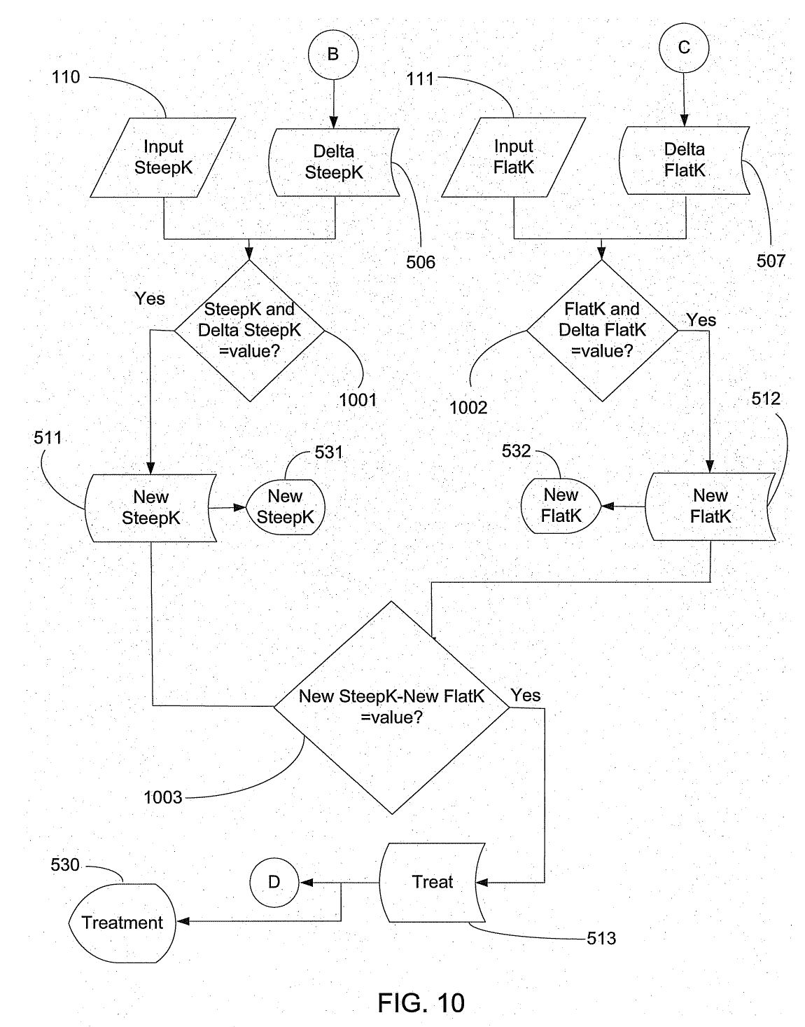

FIG. 10 illustrates determining How-Much-to-Treat 510 a patient's astigmatism induced by phacoemulsification in accordance with the present design. In this arrangement, the present design may calculate the following intermediate values: New SteepK 511, New FlatK 512, and Treat 513 and may present these values relating patient information to the surgeon via a GUI as Treatment 530, New SteepK 531, and New FlatK 532.

FIG. 10 illustrates using SteepK 110 and FlatK 111, as input provided by the user, and Delta SteepK 506 and Delta FlatK 507 as data input values, previously determined by LRI calculator utility 105, to generate values New SteepK 511, New FlatK 512, and Treat 513. New SteepK 511 may be computed by evaluating SteepK 110 and Delta SteepK 506 at decision point 1001. If decision point 1001 represents SteepK 110 and Delta SteepK 506 having values, then the present design may set New SteepK 511 equal to the quantity SteepK minus Delta SteepK. If decision point 1001 does not represent SteepK 110 and Delta SteepK 506 having values, then an error condition exists (not shown).

New FlatK 512 is determined by evaluating FlatK 111 and Delta FlatK 507 at decision point 1002. If FlatK 111 and Delta FlatK 507 have values, the system sets New FlatK 512 equal to FlatK minus Delta FlatK. If FlatK 111 and Delta FlatK 507 do not have values, then an error exists (not shown).

Treat 513 is assessed at point 1003 based on New SteepK 511 and New FlatK 512. If New SteepK minus New FlatK produces a value at decision point 1003, then Treat 513 is equal to the absolute value of (New SteepK minus New FlatK). If the formula does not produce a value, then an error condition exists (not shown).

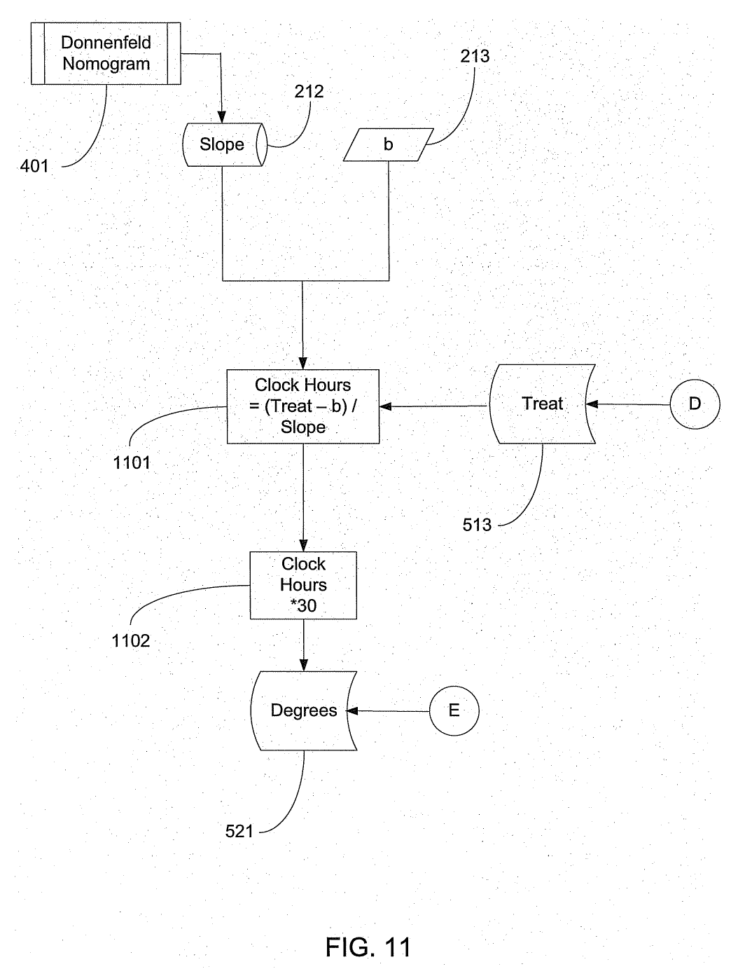

FIG. 11 illustrates calculating an intermediate value for Degrees 521 for Incisions-on-a-Steep-Axis 520 in the phacoemulsification-induced condition. The present design may generate values, i.e. incision angles, for Clock Hours and Degrees 521. FIG. 11 illustrates using the Slope 212 obtained from the Donnenfeld Nomogram 401, user input parameter ‘b’ 213, and Treat 513 as input to generate a value for Degrees 521. The LRI calculator utility 105 may determine Clock Hours at point 1101 according to the following formula:

Clock Hours=(Treat−b)/Slope (13)

### Where

‘b’ 213 is a constant, such as −0.33333333 (y-axis intercept for the linear formula of the Donnenfeld Nomogram);
Treat is the Treat 513 value previously generated; and
Slope 212 is the Largest Astigmatism Value minus Smallest Astigmatism Value at point 1201 divided by the Largest Total Incision Clock Hours (Nomogram) minus Smallest Total Incision Hours (Nomogram) 1202 (from Donnenfeld Nomogram in FIG. 12).

The LRI calculator utility 105 may determine Degrees 521 at point 1102, according to the following formula:

Degrees=Clock Hours*30 (14)

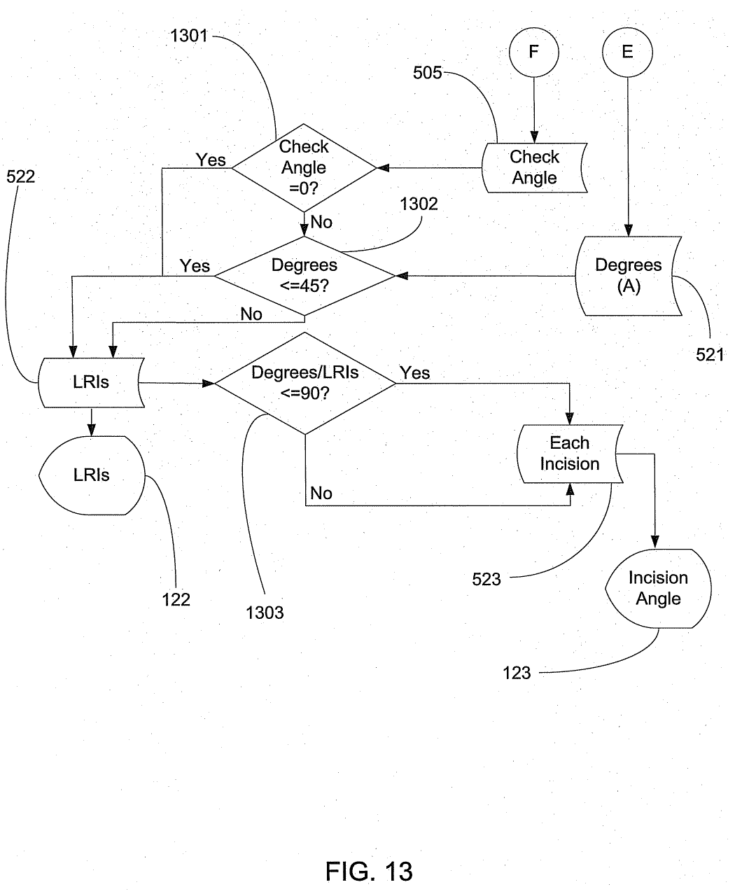

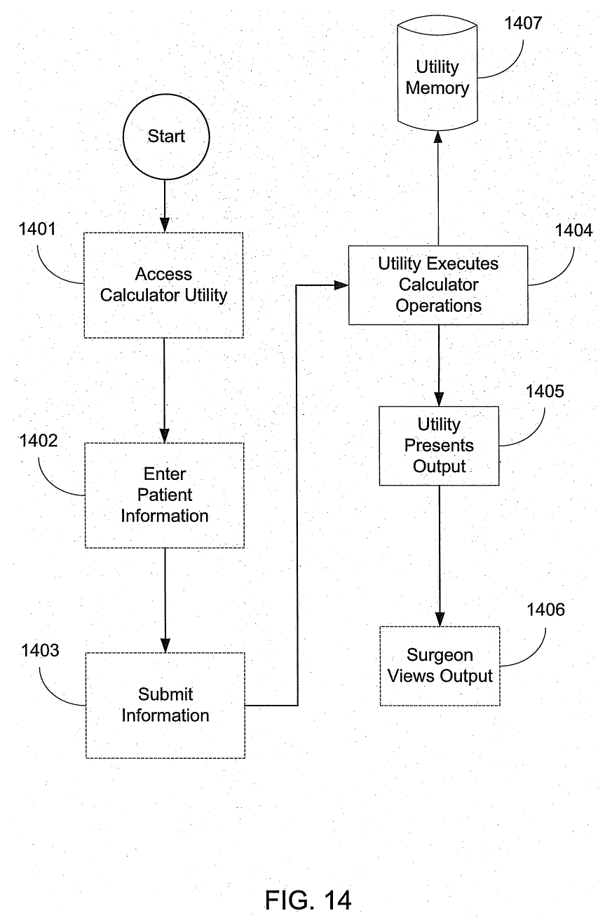

FIG. 13 illustrates LRI calculator utility 105 configured to use Check Angle 505 and Degrees 521 as input to Incisions-on-a-Steep-Axis 520 formulas, which provides the surgeon or user with the resultant number of LRIs 122 and associated Incision Angle 123 for use in performing corrective ocular surgery to treat the patient's condition in accordance with the present design.

In order to calculate the desired output values LRI calculator utility 105 may employ previously determined values for Check Angle 505 and Degrees 521. FIG. 13 illustrates a two-part decision process for calculating the total number of LRIs 522. The first involves evaluating Check Angle 505 at decision point 1301 to generate the number of LRIs. If decision point 1301 is equal to zero, then the present design may set the number of LRIs to equal one. If decision point 1301 is not equal to zero, then the present design employs a second decision at point 1302. The second decision evaluates Degrees 521. If the value found at decision point 1302 is less than or equal to 45 degrees, then LRIs 522 are set to one. If the value at decision point 1302 is greater than 45, LRIs 522 at point 1302 are set to two incisions. Other values and angles may be employed.

The LRI calculation utility 105 may determine Each Incision 523 by evaluating a ratio of Degrees divided by LRIs at decision point 1303. If the ratio at decision point 1303 is less than or equal to 90 degrees, then the Each Incision 523 is set equal to (Degrees divided by LRIs). If the ratio at decision point 1303 is greater than 90 degrees, then Each Incision 523 is set to 90 degrees.

The LRI calculator utility 105 may determine a value for Astigmatism and present the result at 1552. Astigmatism is set equal to New SteepK 511 minus New FlatK 512 for the induced phacoemulsification configuration. The present design may round the resulting astigmatism value to the next quarter (in diopters). The LRI calculator utility 105 may present patient information to the surgeon or a user, via a GUI, including but not limited to the number of LRIs 122 and associated Incision Angle 123. The system may present data and information via a graphical user interface (GUI) to show marks or indicate each incision location and may superimpose these markings over a real-time image of the patient's eye. A surgeon may use the information presented by the system to ascertain the amount of correction necessary to mitigate the patient's eye condition while performing an ocular procedure or surgery.

As described above, the present design may be configured to allow only one LRI to be performed with on-axis phacoemulsification. However, in the off-axis phacoemulsification arrangement the present design may be configured to allow two LRIs to be performed. In addition, the present design may prevent an LRI from being marked or positioned on top of the existing phacoemulsification incision.

### User Interface

FIG. 14 is an example of operational activity flow that may be supported by a graphical user interface device in accordance with an aspect of the present design. One example of such a graphical interface includes, but is not limited to, a browser enabled device 101 and may comprise a personal computer supporting an input device 102 and output device 103 as previously illustrated in FIG. 1. The graphical user interface device may allow an operator/user to provide operational control for the LRI calculation system 100. The user interface device may include but is not limited to a touch screen monitor, mouse, keypad, foot pedal switch, and/or a computer monitor. The personal computer may include memory at point 1407 that may be configured to store, and subsequently retrieve, data generated and obtained during the operation of the LRI calculator utility 105. The utility memory 1407 may be resident within the personal computer, for example a hard drive or RAM, or realized using external devices, such as a memory stick or floppy drive, and/or an attached software system.

The surgeon or other medical practitioner or even the patient or other individual may use a personal computer to access an Internet enabled embodiment of the present design at point 1401. Accessing an Internet enabled application, or, software utility, should be well understood by those skilled in the art. Information relating the patient's present condition and other information, for example surgeons name, operating room number, etc. may be entered by the operator/user at point 1402. When the operator/user completes entering the patient information, the operator/user may submit the information for use by the LRI calculation system 100 in accordance with the present design and may execute LRI calculator utility 105 operations at point 1403. The calculator system 100 may execute calculator utility operations at point 1404 to determine the number of LRIs and associated incision Angle based on the information supplied as input at point 1402. The calculator system 100 may present the results obtained as output at point 1405 for the operator's/user's review. The calculated output results available for presentation may include but are not limited to Flat Meridian K at 1551, New SteepK 511, New FlatK 512, Astigmatism 1201, Treat 513, number of LRI incisions and each associated incision angle.

The surgeon may view the results, presented at point 1406, using the browser-based device providing the GUI. The information generated by the present design may facilitate the surgeon in determining the best approach to completing the patient's ocular procedure. The LRI calculator utility 105 may save the resulting LRI output, associated intermediate values and user provided input values, and other patient data in a database at point 1407 for retrieval at a later time. The LRI calculation system 100 may be optionally configured to communicate the output values to another system arranged to accept LRI parameters values as input (not shown). The present design may be configured to send the results to a hardcopy printer, or other output device, for use by the surgeon.

FIG. 15 illustrates an example of a GUI based user interface for use in operating LRI calculation system 100 in accordance with an aspect of the present design. The GUI based user interface may enable operators/users to input data and obtain output results using a browser based device, such as personal computer, personal digital assistant, WEB enable cellular device, or other browser based device suitable for displaying the LRI calculation system 100 results. FIG. 15A illustrates an exemplary graphical user interface that may allow an operator/user to input patient biometric data 1500 for processing by the LRI calculation system 100. The operator may enter doctor and patient information at point 1501 and indicate eye selection at 1502, either OD—Right or OS—left.

Patient keratometry measurement readings may be entered at point 1503.

The placement of the LRI should be customized to the topography. In cases of asymmetric astigmatism, the LRI in the steepest axis can be elongated slightly and then shortened the same amount in the flatter of the 2 steep axes. Paired LRIs do not have to be made in the same meridian. If the topography reveals non-orthogonal astigmatism, each LRI is placed at the steepest portion of the bow tie.

Patients with low (<1.5 D) against-the-rule astigmatism (1800) receive only a single LRI in the steep meridian, placed opposite to the cataract incision. However, if astigmatism is greater than 1.5 D, a pair of LRIs must be used. In against-the-rule astigmatism cases, one pair of LRIs may be incorporated into the cataract incision. The length of the LRI is not affected by the presence of the cataract incision.

In addition, the present design may provide a check box, radio button or other mechanism as part of the user interface, design that may enable the operator/user to select whether or not the LRI will be done along with a proposed phacoemulsification incision at point 1504. In the induced-phacoemulsification situation information relating the phaco incision including Surgically Induced Cylinder and Incision Location may be entered by the operator/user at point 1504.

Finally, the operator, user, or surgeon may enter the estimated surgically induced cylinder and the location of the cataract surgery incision.

An operator/user may select to ‘continue’ when ready to perform LRI calculations at point 1505, or may select to ‘revise’ the information by selecting ‘reset’ at point 1505. The operator/user may select to present the results at point 1506 using a scale marked in Degrees or a scale indicating Clock Hours overlaid on top of an image of the patient's eye. The present design input GUI screen may present an image of the patient's eye at point 1507.

FIG. 15B illustrates an exemplary graphical user interface for presenting a patient's results as output generated by executing the utilities operations at point 1404 in accordance with an aspect for the present invention. An example output GUI screen 1550 is illustrated in FIG. 15B. The output GUI screen 1550 is not limited to the example illustrated in FIG. 15B and may present a graphical representation and other data superimposed over an actual image of the patients eye, for example as an image overlay on a separate graphical presentation layer, as illustrated in FIG. 15B.

FIG. 153 illustrates an exemplary graphical user interface 1550 for presenting calculation results as output in accordance with an aspect for the present invention. The example GUT screen illustrated in FIG. 15B may be suitable for use in the phacoemulsification-induced arrangement and the without phacoemulsification arrangement. The present design may display a summary of the information, at point 1551 previously provided as input, as part of the output display result presentation. The LRI calculation system 100 results may be presented as part of the output GUI screen at point 1552, and may include but is not limited to presenting values for: Steep Meridian K, Flat meridian K, New SteepK, New FlatK, Astigmatism, Treatment, number of LRI incisions, and each incision angle. Steep Meridian K provides the location of the astigmatism axis, where Flat Meridian K is calculated based on the assumption that the flat axis is 90 degrees rotated from the steep axis.

In addition, the output GUT screen may display an image of the patient's eye 1560, the recommend phacoemulsification location 1561, each LRI incision required 1562, steep axis 1563 and flat axis 1564, and may show on-axis and off-axis phacoemulsification and the resulting phacoemulsification wound when applicable on the graphical user interface. The GUI display may show, for the recommended phacoemulsification location either operating on steep axis for astigmatism up to 2 diopters, or recommend operating on flat axis for up to 3 diopters of astigmatism. Although illustrated as a GUI in FIG. 15, the LRI calculation system 100 may receive information relating patient's biometrics and output results generated using a non-GUI enabled interface such as a text based cellular telephone or similar device.

Arranged in the foregoing manner, the apparatus and method disclosed herein may generate highly accurate and repeatable LRI calculations enabling a surgeon to precisely locate each required incision site by depicting where and how long to make limbal relaxing incisions for reducing a patient's astigmatism via a user interface. The calculations may involve a nomogram, patient keratometry measurement readings, and other factors such as if phacoemulsification-induced astigmatism involved, required to execute operations sufficient for an operator/user to determine how and where to correct a patient's eye aliment or condition superimposed on a real-time image of the patient's eye. The operable range of the present design may enable surgeons to perform eye procedures for a greater range of patient keratometry measurement values on the eye for cord length than achievable with current manual calculation methods. Furthermore, the present design may reduce or eliminate inaccuracies exhibited by current manual calculation based techniques.

The design presented herein and the specific aspects illustrated are meant not to be limiting, but may include alternate components while still incorporating the teachings and benefits of the invention. While the invention has thus been described in connection with specific embodiments thereof, it will be understood that the invention is capable of further modifications. This application is intended to cover any variations, uses or adaptations of the invention following, in general, the principles of the invention, and including such departures from the present disclosure as come within known and customary practice within the art to which the invention pertains.

The foregoing description of specific embodiments reveals the general nature of the disclosure sufficiently that others can, by applying current knowledge, readily modify and/or adapt the system and method for various applications without departing from the general concept. Therefore, such adaptations and modifications are within the meaning and range of equivalents of the disclosed embodiments. The phraseology or terminology employed herein is for the purpose of description and not of limitation.

What is claimed is:

1. A system configured to perform ocular incision calculations, comprising:
a computing device comprising:
a Limbal Relaxation Incision (LRI) calculator utility; and
a user interface configured to obtain information from a user and interface with the LRI calculator utility by to present the user with at least one potential incision;
wherein the LRI calculator utility is configured to calculate LRIs based on a nomogram relating astigmatism conditions to incisions.

2. The system of claim 1, wherein said user interface comprises a graphical user interface (GUI) for input and output of patient biometric data for LRI calculator utility operations.

3. The system of claim 1, wherein said user interface comprises a text based interface for receipt of patient biometric data employed in LRI calculator utility operations.

4. The system of claim 1, wherein the nomogram is a Donnenfeld Nomogram.

5. The system of claim 1, wherein the LRI calculator utility operations determine at least one incision and incision angle.

6. The system of claim 5, wherein the incision and incision angle are determined without phacoemulsification induced astigmatism.

7. The system of claim 5, wherein said LRI and Incision Angle are determined with phacoemulsification induced astigmatism.

8. The system of claim 1, wherein the computing device further comprises a memory configured to store LRI calculation data and values employable by said LRI calculator utility.

9. A method configured for execution on a general purpose computing device for calculating Limbal Relaxing Incision (LRIs), comprising:
inputting an individual's biometric information;
determining LRI parameters from said individual's biometric information and applying data from a nomogram relating astigmatism conditions and LRIs to determine at least one LRI and one incision angle; and
presenting the LRI and incision angle to a user.

10. The method of claim 9, wherein said biometric information includes the individual's keratometry measurement readings.

11. The method of claim 9, wherein said nomogram is a Donnenfeld Nomogram.

12. The method of claim 11, wherein said LRI and Incision Angle are determined without phacoemulsification induced astigmatism.

13. The method of claim 11, wherein said LRI and Incision Angle are determined with phacoemulsification induced astigmatism.

14. The method of claim 9, wherein presenting occurs using graphical user interface configured to show the LRI and angle superimposed over a visual representation of an eye.

15. A method configured for execution on a general purpose computing device for calculating ocular incisions to address astigmatism in an eye, comprising:
providing biometric information;
determining an incision location and angle based on said biometric information and a nomogram relating astigmatism conditions and incision conditions; and
presenting the incision location and angle to a user.

16. The method of claim 15, wherein said biometric information includes keratometry measurement readings.

17. The method of claim 15, wherein said nomogram is a Donnenfeld Nomogram.

18. The method of claim 17, wherein said incision location and angle are determined for a situation without phacoemulsification induced astigmatism.

19. The method of claim 17, wherein said incision location and angle are determined for a situation with phacoemulsification induced astigmatism.

20. The method of claim 15, wherein presenting occurs using graphical user interface configured to show the incision location and angle superimposed over a visual representation of an eye.
